(19) United States
(12) Patent Application Publication
(10) Pub. No.: US 2023/0113511 A1
(43) Pub. Date: Apr. 13, 2023

(54) USE OF TMAVA ANTAGONIST AND SYSTEM FOR DIAGNOSING FATTY LIVER OR HEART FAILURE

(71) Applicants: Lemin ZHENG, Fuzhou, Fujian (CN); ProgLead, Inc, Fuzhou, Fujian (CN)

(72) Inventors: Lemin ZHENG, Fuzhou, Fujian (CN); Daowen WANG, Wuhan, Hubei (CN); Xiangbao MENG, Beijing (CN); Mingming ZHAO, Beijing (CN); Chenze LI, Wuhan, Hubei (CN)

(21) Appl. No.: 17/904,702

(22) PCT Filed: Feb. 26, 2021
(86) PCT No.: PCT/CN2021/078272

§ 371 Date: Dec. 8, 2022

(30) Foreign Application Priority Data
Feb. 20, 2020 (CN) 202010105147.2

(51) Int. Cl. A61K 31/205; A61P 1/16; A61P 9/00; G01N 30/72

(52) U.S. Cl. CPC A61K 31/205; A61P 1/16; A61P 9/00; G01N 30/7233; G01N 2030/027

(57) ABSTRACT

Use of a N,N,N-trimethyl-5-aminopentanoic acid antagonist in the preparation of a medicament for treating fatty liver or heart failure and a system for diagnosing fatty liver or heart failure. New treatment regimens are provided for fatty liver or heart failure. By antagonizing TMAVA, the levels of triglyceride and free fatty acids in the plasma and liver of a patient can be reduced, and at the same time, the inhibition effect of TMAVA on the activity of γ-isopropylbetaine hydroxylase can also be eliminated, and the weakening of free fatty acid mitochondrial β-oxidation caused by the decrease of endogenous carnitine synthesis is avoided. It is thus determined that reducing the ectopic accumulation of fatty acids in the liver and heart is clearly effective in treating fatty liver or heart failure.

### CROSS REFERENCE TO RELATED APPLICATIONS

The present application is a U.S. National Phase of International Application No. PCT/CN2021/078272 entitled “USE OF TMAVA ANTAGONIST AND SYSTEM FOR DIAGNOSING FATTY LIVER OR HEART FAILURE,” and filed on Feb. 26, 2021. International Application No. PCT/CN2021/078272 claims priority to Chinese Patent Application No. 202010105147.2 filed on Feb. 20, 2020. The entire contents of each of the above-listed applications are hereby incorporated by reference for all purposes.

### TECHNICAL FIELD

The present disclosure relates to the field of biotechnology, in particular to use of a N,N,N-trimethyl-5-aminovaleric acid antagonist in the preparation of a medicament for treating fatty liver or heart failure and a system for diagnosing fatty liver or heart failure.

### BACKGROUND

Non-alcoholic fatty liver disease (NAFLD) refers to a clinicopathological syndrome mainly characterized by excessive deposition of fat in liver cells caused by the exclusion of alcohol and other definite liver damage factors, including simple fatty liver (SFL), non-alcoholic steatohepatitis (NASH) and related liver cirrhosis. With the global trend of obesity and related metabolic syndromes thereof, NAFLD has become an important cause of chronic liver disease in developed countries such as Europe and the United States and in wealthy areas of China. The prevalence of NAFLD in ordinary adults is 10%-30%.

In addition to directly leading to decompensated liver cirrhosis, hepatocellular carcinoma and recurrence of transplanted liver, NAFLD can also affect the progression of other chronic liver diseases and be involved in the pathogenesis of type 2 diabetes and atherosclerosis. Malignant tumors, arteriosclerotic cardiovascular and cerebrovascular diseases, and liver cirrhosis related to metabolic syndromes are important factors affecting the quality of life and life expectancy of patients with non-alcoholic fatty liver diseases. Therefore, NAFLD is a new challenge in the field of contemporary medicine, and the harm of NAFLD to human health will continue to increase in the near future.

However, despite the high incidence and severity of NAFLD, effective treatments for NAFLD still lack.

### SUMMARY

The objective of the present disclosure is to provide use of a N,N,N-trimethyl-5-aminovaleric acid antagonist in the preparation of a medicament for treating fatty liver or heart failure and a system for diagnosing fatty liver or heart failure.

In order to achieve the above objective, the present disclosure provides use of a N,N,N-trimethyl-5-aminovaleric acid antagonist in the preparation of a medicament for treating fatty liver or heart failure.

Optionally, the N,N,N-trimethyl-5-aminovaleric acid antagonist includes at least one of a reagent capable of inhibiting the activity of N,N,N-trimethyl-5-aminovaleric acid, a reagent capable of eliminating N,N,N-trimethyl-5-aminovaleric acid, a reagent capable of reducing or preventing the production of N,N,N-trimethyl-5-aminovaleric acid, and a reagent capable of promoting the metabolism of N,N,N-trimethyl-5-aminovaleric acid in vivo.

Optionally, the fatty liver includes non-alcoholic fatty liver disease; and the heart failure is heart failure caused by myocardial hypertrophy.

Optionally, the non-alcoholic fatty liver disease includes high-fat diet-induced non-alcoholic fatty liver disease; and the myocardial hypertrophy includes high-fat diet-induced myocardial hypertrophy.

The present disclosure further provides a system for diagnosing fatty liver or heart failure. The system includes a test device, a computing device and an output device; the test device includes a sampler and a tester, the sampler is used to collect a sample to be tested, and the tester is used to test the concentration of N,N,N-trimethyl-5-aminovaleric acid in the sample to be tested; the computing device includes a memory and a processor, the memory stores a computer program, and the processor is configured to execute the computer program stored in the memory to implement the following determination:

If the test result of the concentration of N,N,N-trimethyl-5-aminovaleric acid in the sample to be tested is 0.576-100 μmol/L, it is determined that the subject corresponding to the sample to be tested is a fatty liver patient or a heart failure patient.

Optionally, the fatty liver includes non-alcoholic fatty liver disease; and the heart failure is heart failure caused by myocardial hypertrophy.

Optionally, the non-alcoholic fatty liver disease includes high-fat diet-induced non-alcoholic fatty liver disease; and the myocardial hypertrophy includes high-fat diet-induced myocardial hypertrophy.

Optionally, the sample to be tested includes a plasma sample or a serum sample.

Optionally, the test device includes a mass spectrometer or a liquid chromatograph-mass spectrometer.

Optionally, the output device is used to output the test result of the test device or the determination result of the computing device, and the output device includes at least one of a display, a printer and an audio output device;

wherein the computing device includes at least one of a computer host, a central processing unit and a network server.

The present disclosure further provides a method for treating fatty liver or heart failure, the method including: reducing the level of N,N,N-trimethyl-5-aminovaleric acid in a treatment target body, the level of N,N,N-trimethyl-5-aminovaleric acid in the treatment target body including the concentration of N,N,N-trimethyl-5-aminovaleric acid in the treatment target body.

Optionally, the reducing the level of N,N,N-trimethyl-5-aminovaleric acid in a treatment target body includes: using a N,N,N-trimethyl-5-aminovaleric acid antagonist for the treatment target, the N,N,N-trimethyl-5-aminovaleric acid antagonist including at least one of a reagent capable of inhibiting the activity of N,N,N-trimethyl-5-aminovaleric acid, a reagent capable of eliminating N,N,N-trimethyl-5-aminovaleric acid, a reagent capable of reducing or preventing the production of N,N,N-trimethyl-5-aminovaleric acid, and a reagent capable of promoting the metabolism of N,N,N-trimethyl-5-aminovaleric acid in vivo.

The present disclosure further provides a method for diagnosing fatty liver or heart failure, the method including: testing the level of N,N,N-trimethyl-5-aminovaleric acid in a treatment target body, the level of N,N,N-trimethyl-5-aminovaleric acid in the treatment target body including the concentration of N,N,N-trimethyl-5-aminovaleric acid in the treatment target body.

Optionally, if the test result of the concentration of N,N,N-trimethyl-5-aminovaleric acid in the treatment target body is 0.576-100 μmol/L, it is determined that the treatment target body has fatty liver or heart failure.

Through the above technical solutions, the present disclosure provides new diagnosis and treatment methods for fatty liver or heart failure, with fast diagnosis speed and good treatment effect.

Other features and advantages of the present disclosure will be described in detail in the following specific embodiments.

### DETAILED DESCRIPTION

Specific embodiments of the present disclosure will be described in detail below. It should be understood that the specific embodiments described herein are merely used for illustrating and interpreting the present disclosure, rather than limiting the present disclosure.

A first aspect of the present disclosure provides use of a N,N,N-trimethyl-5-aminovaleric acid antagonist in the preparation of a medicament for treating fatty liver or heart failure.

The inventors of the present disclosure found that the levels of N,N,N-trimethyl-5-aminovaleric acid (TMAVA) in patients with non-alcoholic fatty liver disease and myocardial hypertrophy were significantly higher than those in normal people. Antibiotic flora inhibition experiments and flora transplantation experiments showed that TMAVA was a metabolite of intestinal flora; radioisotope tracer experiments showed that TMAVA was produced by trimethyl lysine under the action of lysine 2-monooxygenase (DavB) and 5-aminovaleramidase (DavA) of intestinal bacteria; and in animal experiments, mice were given TMAVA intervention, and it was found that TMAVA can promote the occurrence of fatty liver and myocardial hypertrophy in mice under high-fat diets, and can also change the composition of mice intestinal flora.

Studies found that TMAVA can promote lipidolysis, resulting in significantly increased levels of triglyceride and free fatty acid in the plasma and liver of patients; at the same time, TMAVA can also competitively inhibit the activity of γ-isopropylbetaine hydroxylase (Bbox), resulting in the decrease of endogenous carnitine synthesis, thus weakening free fatty acid mitochondrial β-oxidation in patients, and promoting ectopic accumulation of fatty acid in the liver and heart of patients. Therefore, TMAVA can be used as a therapeutic target in patients with non-alcoholic fatty liver disease and myocardial hypertrophy.

TMAVA is produced by the metabolism of intestinal flora. Literature reported that lysine produced pentaaminovaleric acid by means of lysine 2-monooxygenase (DavB) and 5-aminovaleramidase (DavA) of microorganisms, so it was speculated whether trimethyllysine can produce trimethylaminovaleric acid by the same enzymatic reaction. Trimethyllysine (TML) is an important precursor for endogenous carnitine synthesis. Mice were given d9-TML by gavage, and the plasma level of d9-TMAVA was found to increase over time. Co-incubation of conventionally raised mice feces with different concentrations of TML also found that the TMAVA level also increased over time and concentration, while sterilized feces had no such effect. Therefore, TML produces TMAVA through a similar pathway under the action of intestinal flora. Furthermore, by constructing recombinant Escherichia coli strains expressing davB and davA genes, TMAVA was time-dependently produced in vitro when purified DavA and DavB proteins were co-incubated with TML. When TML was added to the drinking water of mice, rising levels of TMAVA were tested in the urine. It was proved that TML was metabolized by intestinal flora to produce TMAVA.

Through the above technical solution, the present disclosure provides treatment regimens for fatty liver or heart failure. By antagonizing TMAVA, the levels of triglyceride and free fatty acid in the plasma and liver of a patient can be reduced, and at the same time, the inhibition effect of TMAVA on the activity of γ-isopropylbetaine hydroxylase can also be eliminated, and the weakening of free fatty acid mitochondrial β-oxidation caused by the decrease of endogenous carnitine synthesis is avoided. It is thus determined that reducing the ectopic accumulation of fatty acid in the liver and heart is clearly effective in treating fatty liver or heart failure.

According to the present disclosure, the species of the N,N,N-trimethyl-5-aminovaleric acid antagonist may be selected in a wide range, for example, the N,N,N-trimethyl-5-aminovaleric acid antagonist may include at least one of a reagent capable of inhibiting the activity of N,N,N-trimethyl-5-aminovaleric acid, a reagent capable of eliminating N,N,N-trimethyl-5-aminovaleric acid, a reagent capable of reducing or preventing the production of N,N,N-trimethyl-5-aminovaleric acid, and a reagent capable of promoting the metabolism of N,N,N-trimethyl-5-aminovaleric acid in vivo. The reagent capable of inhibiting the activity of N,N,N-trimethyl-5-aminovaleric acid weakens the activity inhibition effect of N,N,N-trimethyl-5-aminovaleric acid on γ-isopropylbetaine hydroxylase, increases the production of endogenous carnitine and reduces lipidolysis by inhibiting the bioactivity of N,N,N-trimethyl-5-aminovaleric acid, thereby controlling fatty acid levels in patients. The reagent capable of eliminating N,N,N-trimethyl-5-aminovaleric acid, the reagent capable of reducing or preventing the production of N,N,N-trimethyl-5-aminovaleric acid, and the reagent capable of promoting the metabolism of N,N,N-trimethyl-5-aminovaleric acid in vivo can also weakens the activity inhibition effect of N,N,N-trimethyl-5-aminovaleric acid on γ-isopropylbetaine hydroxylase in patients, reduce lipidolysis and increase the production of endogenous carnitine by reducing the levels of N,N,N-trimethyl-5-aminovaleric acid in patients and reducing the accumulation of N,N,N-trimethyl-5-aminovaleric acid in patients, thereby controlling fatty acid levels in patients.

Preferably, the fatty liver may include non-alcoholic fatty liver disease; and the heart failure may be heart failure caused by myocardial hypertrophy. Further preferably, the non-alcoholic fatty liver disease may include high-fat diet-induced non-alcoholic fatty liver disease; and the myocardial hypertrophy may include high-fat diet-induced myocardial hypertrophy.

A second aspect of the present disclosure provides a system for diagnosing fatty liver or heart failure. The system includes a test device, a computing device and an output device; the test device includes a sampler and a tester, the sampler is used to collect a sample to be tested, and the tester is used to test the concentration of N,N,N-trimethyl-5-aminovaleric acid in the sample to be tested; the computing device includes a memory and a processor, the memory stores a computer program, and the processor is configured to execute the computer program stored in the memory to implement the following determination:

If the test result of the concentration of N,N,N-trimethyl-5-aminovaleric acid in the sample to be tested is 0.576-100 μmol/L, it is determined that the subject corresponding to the sample to be tested is a fatty liver patient or a heart failure patient.

Specifically, a sample to be tested is collected from a subject who is suspected to be a fatty liver patient and tested, and if the test result of the concentration of N,N,N-trimethyl-5-aminovaleric acid in the sample to be tested is 0.647-100 μmol/L, it is determined that the subject corresponding to the sample to be tested is a fatty liver patient. A sample to be tested is collected from a subject who is suspected to be a heart failure patient and tested, and if the test result of the concentration of N,N,N-trimethyl-5-aminovaleric acid in the sample to be tested is 0.576-100 μmol/L, it is determined that the subject corresponding to the sample to be tested is a heart failure patient.

Through the above technical solution, the system of the present disclosure is easy to use, and can realize rapid diagnosis of fatty liver or heart failure.

Preferably, the fatty liver includes non-alcoholic fatty liver disease; and the heart failure is heart failure caused by myocardial hypertrophy. Further preferably, the non-alcoholic fatty liver disease includes high-fat diet-induced non-alcoholic fatty liver disease; and the myocardial hypertrophy includes high-fat diet-induced myocardial hypertrophy.

According to the present disclosure, the sample to be tested may be selected in a wide range, and any sample that can reflect the level of N,N,N-trimethyl-5-aminovaleric acid in a patient can be used as the sample to be tested in the present disclosure, for example, the sample to be tested may be a plasma sample or a serum sample.

According to the present disclosure, the test device may be selected in a wide range, and any test device capable of accurately and quantitatively testing the N,N,N-trimethyl-5-aminovaleric acid in the sample to be tested can be used in the present disclosure, for example, the test device may be a mass spectrometer or a liquid chromatograph-mass spectrometer.

Preferably, the output device is configured to output the test result of the test device or the determination result of the computing device, and the output device includes at least one of a display, a printer and an audio output device; and the computing device includes at least one of a computer host, a central processing unit and a network server.

A third aspect of the present disclosure provides a method for treating fatty liver or heart failure, the method including: reducing the level of N,N,N-trimethyl-5-aminovaleric acid in a treatment target body, the level of N,N,N-trimethyl-5-aminovaleric acid in the treatment target body including the concentration of N,N,N-trimethyl-5-aminovaleric acid in the treatment target body.

Optionally, the reducing the level of N,N,N-trimethyl-5-aminovaleric acid in a treatment target body includes: using a N,N,N-trimethyl-5-aminovaleric acid antagonist for the treatment target, the N,N,N-trimethyl-5-aminovaleric acid antagonist including at least one of a reagent capable of inhibiting the activity of N,N,N-trimethyl-5-aminovaleric acid, a reagent capable of eliminating N,N,N-trimethyl-5-aminovaleric acid, a reagent capable of reducing or preventing the production of N,N,N-trimethyl-5-aminovaleric acid, and a reagent capable of promoting the metabolism of N,N,N-trimethyl-5-aminovaleric acid in vivo.

A fourth aspect of the present disclosure provides a method for diagnosing fatty liver or heart failure, the method including: testing the level of N,N,N-trimethyl-5-aminovaleric acid in a treatment target body, the level of N,N,N-trimethyl-5-aminovaleric acid in the treatment target body including the concentration of N,N,N-trimethyl-5-aminovaleric acid in the treatment target body.

Optionally, if the test result of the concentration of N,N,N-trimethyl-5-aminovaleric acid in the treatment target body is 0.576-100 μmol/L, it is determined that the treatment target body has fatty liver or heart failure.

Preferably, a sample to be tested is collected from a subject who is suspected to be a fatty liver patient and tested, and if the test result of the concentration of N,N,N-trimethyl-5-aminovaleric acid in the sample to be tested is 0.647-100 μmol/L, it is determined that the subject corresponding to the sample to be tested is a fatty liver patient. A sample to be tested is collected from a subject who is suspected to be a heart failure patient and tested, and if the test result of the concentration of N,N,N-trimethyl-5-aminovaleric acid in the sample to be tested is 0.576-100 μmol/L, it is determined that the subject corresponding to the sample to be tested is a heart failure patient.

A fifth aspect of the present disclosure provides use of a reagent for testing N,N,N-trimethyl-5-aminovaleric acid in the preparation of a kit for diagnosing fatty liver or heart failure.

A sixth aspect of the present disclosure provides a kit for diagnosing fatty liver or heart failure, the kit including a reagent for testing N,N,N-trimethyl-5-aminovaleric acid.

The present disclosure is further described by the following examples, but the present disclosure is not limited thereby. The raw materials, reagents, instruments, and equipment involved in the examples of the present disclosure can be purchased from the market unless otherwise specified.

### EXAMPLE 1

### Preparation of N,N,N-trimethyl-5-aminovaleric acid (TMAVA)

20 mmol of dimethylaminobutyrate hydrochloride was gradually dissolved in dichloromethane, and then 22 mmol of trimethylamine alcohol (Thermo Fisher) was added dropwise at room temperature; the obtained mixture was stirred overnight, then the solvent was removed by filtration under reduced pressure, and the filter residue was collected; the filter residue (deep brown powder) was dissolved in water to form a 10 mmol/L solution, and 15 mmol of hydrochloric acid (Thermo Fisher) was added; the obtained solution was heated to 70° C. overnight and then continuously heated to 100° C. for 12 h, the solvent was removed by filtration under reduced pressure, the filter residue was collected and the collected filter residue was added to acetone for refluxing 2 h; the solution was cooled to room temperature and filtered, the filter residue was collected and dried under reduced pressure at 40° C. for 12 h to obtain a white solid of N,N,N-trimethyl-5-aminovaleric acid (TMAVA), and the TMAVA was stored in a polyethylene container.

### Example 2

### Production of TMAVA from TML by Metabolism in Intestinal Flora

First, normal mice were given antibiotics for 4 weeks to inhibit the intestinal flora of the mice, and it was found that the TMAVA level of the antibiotic-treated mice plasma was significantly reduced. The antibiotic-treated mice were then placed in conventional cages with normal (non-sterile) mice to colonize the intestinal flora. The TMAVA level in the “recolonized” mice plasma was significantly increased after raising in the conventional cages for four weeks or fecal transplantation for one week. At the same time, the germ-free mice also had only a trace amount of TMAVA in plasma, feces and urine, which was significantly lower than that of the normal mice. The TMAO was also significantly reduced in the germ-free mice. Therefore, the TMAVA was produced by means of the intestinal flora. The mice were given d9-TML by gavage, and the plasma level of d9-TMAVA was found to increase over time. Co-incubation of conventionally raised mice feces with different concentrations of TML also found that the TMAVA level also increased over time and concentration, while sterilized feces had no such effect. In addition, recombinant Escherichia coli strains expressing davB and davA genes were constructed, and when purified DavA and DavB proteins were co-incubated with TML, TMAVA was time-dependently produced in vitro. When the TML was added to the drinking water of mice, the TMAVA level in urine increased. These results showed that the TML was metabolized by intestinal flora to produce TMAVA.

### Example 3

TMAVA promoted the occurrence of high-fat diet-induced fatty liver and myocardial hypertrophy in mice.

Mice in an experimental group were given a high-fat diet and drinking water dissolved with 0.325% (mass/volume %) TMAVA for 8 weeks; and mice in a control group were given a high-fat diet and normal drinking water. After 8 weeks, the mice in the experimental group and the control group were sacrificed and tested. The test results showed that the concentration of TMAVA in the plasma and various tissues of the mice in the experimental group increased by 10 times compared with the mice in the control group; the liver and heart of the mice in the experimental group were significantly larger than those of the mice in the control group; the cardiac systolic function of the mice in the experimental group was significantly reduced; the concentration of aspartate aminotransferase in the plasma of the mice in the experimental group was higher than that of the mice in the control group; in a fasting state, the content of triglyceride in the plasma of the mice in the experimental group was significantly higher than that of the mice in the control group; the liver and heart tissues of the mice were frozen, then sectioned, and stained with oil red O, showing that the lipid droplet content in the liver and heart tissues of the mice in the experimental group was higher than that of the mice in the control group; and the level of triglyceride in the liver and heart tissues of the mice in the experimental group was significantly higher than that of the mice in the control group.

It can be seen that TMAVA can promote the occurrence of high-fat diet-induced fatty liver and myocardial hypertrophy in mice.

### Example 4

TMAVA can competitively inhibit the activity of γ-isopropylbetaine hydroxylase (Bbox).

Endogenous carnitine synthesis played an important role in physiological dynamic equilibrium. A carnitine precursor (γ-BB) was hydroxylated by y-isopropylbetaine hydroxylase (Bbox) to produce carnitine.

Mice in an experimental group were given a high-fat diet, drinking water dissolved with 0.325% (mass/volume %) TMAVA, and d9-γ-BB; and mice in the control group were given a high-fat diet, normal drinking water, and d9-γ-BB. After 8 weeks, the mice in the experimental group and the control group were sacrificed and tested. The level of d9 carnitine in the mice of the experimental group was significantly lower than that of the mice of the control group. Bbox was immobilized on a graphene surface and contacted with TMAVA, and it was found that TMAVA can bind to Bbox in a similar way as γ-BB to Bbox. The binding affinity of Bbox and TMAVA was tested by means of surface plasmon resonance, and the separation rate coefficient constant was 9.8 μM, indicating that TMAVA has affinity to Bbox. The normal mice and the TMAVA-treated mice were given d9-carnitine by gavage, and it was found that the level of d9-carnitine in the plasma of the TMAVA-treated mice was significantly lower than that of the normal mice, indicating that TMAVA can affect carnitine absorption.

It can be seen that TMAVA can competitively bind to Bbox to affect γ-BB and Bbox, thereby inhibiting the bioactivity of Bbox and reducing the synthesis of carnitine, and TMAVA can also affect the absorption of carnitine in vivo.

### Example 5

TMAVA can promote lipidolysis and increase the level of free fatty acid.

Mice in an experimental group were given a high-fat diet and drinking water dissolved with 0.325% (mass/volume %) TMAVA; and mice in a control group were given a high-fat diet and normal drinking water. After 8 weeks, the mice in the experimental group and the control group were sacrificed and tested. The test showed that the weight of the mice in the experimental group decreased by 30% compared with the mice in the control group; histological analysis showed that the percentage of small fat cells in the subcutaneous fat of the mice in the experimental group increased, the percentage of large and medium-sized fat cells decreased, the size of fat cells mainly depended on the accumulation of triglyceride, and the accumulation of triglyceride in the small fat cells was lower; the level of free fatty acid in the blood of the mice in the experimental group was higher than that of the mice in the control group.

It can be seen that TMAVA can promote lipidolysis and increase the level of free fatty acid.

### Example 6

BBOX knockout aggravated fatty liver and myocardial hypertrophy.

After 8 weeks of HFD diet, the liver and heart of BBOX knockout mice were significantly heavier, and the BBOX knockout mice appeared paler than normal mice. The cardiac systolic function of the BBOX knockout mice was significantly reduced; and the levels of triglyceride and FFA in the plasma were increased. Oil red O staining results of frozen liver and heart sections showed that the liver and heart of the BBOX knockout mice had more lipid droplet deposition than the normal mice. Liver HE staining results of the BBOX knockout mice showed increased vacuolation, which was consistent with what happened with TMAVA treatment. Due to the reduced carnitine level, the species of acyl carnitine in the plasma were also significantly reduced, and liver and heart FAO were significantly inhibited in the BBOX knockout mice.

It can be seen that BBOX knockout will aggravate fatty liver and myocardial hypertrophy.

### Example 7

Supplementation of exogenous carnitine can reverse TMAVA-induced fatty liver and myocardial hypertrophy in mice.

Mice were given a high-fat diet and drinking water containing 0.325% (mass/volume %) TMAVA; at the same time, one group was given TMAVA with 0.325% (mass/volume %) carnitine added to the drinking water (carnitine supplementation group). After 8 weeks, the experiment found that after carnitine was supplemented, the aspartate aminotransferase of the mice in the carnitine supplementation group decreased to 50 U/L; the level of triglyceride in the plasma of the mice in the carnitine supplementation group decreased by 50%; the level of free fatty acid in the plasma of the mice in the carnitine supplementation group decreased; the weight of the heart of the mice in the carnitine supplementation group was significantly reduced; the liver of two groups of mice was frozen, sectioned, and stained with oil red O, showing that the lipid content in the liver tissues of the mice in the carnitine supplementation group decreased; the content of triglyceride in the liver tissues of the mice in the carnitine supplementation group decreased by 20%; and the cardiac systolic function of the mice in the carnitine supplementation group was significantly improved.

It can be seen that the supplementation of exogenous carnitine can reverse TMAVA-induced fatty liver and myocardial hypertrophy in mice, indicating that the occurrence of fatty liver and myocardial hypertrophy in mice was related to the reduction of carnitine.

To sum up, TMAVA can promote lipidolysis, resulting in significantly increased levels of triglyceride and free fatty acid in the plasma and liver of patients; at the same time, TMAVA can also competitively inhibit the activity of γ-isopropylbetaine hydroxylase (Bbox), resulting in the decrease of endogenous carnitine synthesis, thus weakening free fatty acid mitochondrial β-oxidation in patients, and promoting ectopic accumulation of fatty acid in the liver of patients to cause fatty liver. Similarly, TMAVA can also be proved to cause myocardial hypertrophy.

### Example 8

This example was used to describe the determination of TMAVA concentration in the plasma of fatty liver patients in the present disclosure.

Source of medical records and sample size: multiple subjects (17-80 years old) from Zhongshan Hospital of Fudan University, excluding the following groups: (1) patients with heart, lung or kidney diseases; (2) patients with a history of alcoholism; and (3) patients with other known liver diseases. 494 healthy subjects and 273 fatty liver patients were ultimately identified by ultrasonography.

Test method: 20 μL of subject's plasma was collected as a sample to be tested, the sample to be tested was put into a 1.5 mL test tube, 80 μL of methanol containing 10 μM of d9-TMAVA was added, the sample was fully shaken and then centrifuged under 12000 g condition, and the supernatant was collected. 70 μL of the supernatant was taken and the concentration of TMAVA was tested by liquid chromatography-mass spectrometry. The liquid chromatograph was an LC-20AD Shimadazu pump system with an SIL-20AXR automatic sampler, the mass spectrometer was API 5500Q-TRAP (AB SCIEX), and the chromatographic column was Luna 5u Silica 100A (2.0*150 mm; Art. No. 00F-4274-BO, Phenomenex). Test conditions included: the flow rate was 0.5 mL/min, the A phase was 0.1% propionic acid+water, the B phase was 0.1% acetic acid+methanol, TMAVA was separated linearly from 2% B to 95% B within 0-5.0 minutes, 95% B was held for 1.0 min, and then the B returned to the gradient of 2%; multiple reaction monitoring (MRM) was used in a positive ion mode for test, the ion pair of TMAVA was 160.1→83, and the ion pair of d9-TMAVA was 169.1→83. The concentration of TMAVA in the plasma of each healthy subject and fatty liver patient was tested by the above method to obtain a test result.

The test results of the concentrations of TMAVA in the plasma of all the healthy subjects and fatty liver patients were subjected to calculation with IVDstatistics software to obtain a cut off value (0.647 μmol/L) of the concentrations of TMAVA in the plasma of the healthy subjects and fatty liver patients.

### Example 9

This example was used to describe the determination of TMAVA concentration in the plasma of heart failure patients in the present disclosure.

Source of medical records and sample size: multiple subjects from Tongji Hospital Affiliated to Tongji Medical College of Huazhong University of Science and Technology, excluding the following groups: (1) patients with obvious valvular heart diseases; (2) patients with severe hepatic insufficiency, and patients with renal insufficiency not related to chronic heart failure; (3) patients with a history of cancer or with formal precancerous lesions in pathological examination and a life expectancy of less than 1 year; (4) patients with untreated second- or third-degree atrioventricular block; and (5) patients with acute myocardial infarction/unstable angina pectoris in the past month. 500 healthy subjects and 500 patients with chronic heart failure were ultimately identified by ultrasonography (decreased ejection fraction or abnormal diffuse wall motion) combined with clinical symptoms and biochemical test (BNP>35 ng/mL or NT-proBNP>125 pg/mL).

Test method: the concentration of TMAVA in the plasma of each healthy subject and chronic heart failure patient was tested by using the test method described in Example 8 to obtain a test result.

The test results of the concentrations of TMAVA in the plasma of all the healthy subjects and chronic heart failure patients were subjected to calculation with IVDstatistics software to obtain a cut off value (0.576 μmol/L) of the concentrations of TMAVA in the plasma of the healthy subjects and chronic heart failure patients.

The preferred embodiments of the present disclosure are described above in detail. However, the present disclosure is not limited to the specific details in the above embodiments. Various simple variations may be made to the technical solutions of the present disclosure within the scope of the technical idea of the present disclosure, and these simple variations fall within the protection scope of the present disclosure.

It should be further noted that the specific technical features described in the above specific embodiments may be combined in any suitable manner without contradiction. In order to avoid unnecessary repetition, the present disclosure will not describe various possible combinations.

Moreover, the various different embodiments of the present disclosure may be combined randomly without deviating from the idea of the present disclosure, and the combinations should also be regarded as the disclosure of the present disclosure.

1. Use of a N,N,N-trimethyl-5-aminovaleric acid antagonist in the preparation of a medicament for treating fatty liver or heart failure.

2. The use according to claim 1, wherein the N,N,N-trimethyl-5-aminovaleric acid antagonist comprises at least one of a reagent capable of inhibiting the activity of N,N,N-trimethyl-5-aminovaleric acid, a reagent capable of eliminating N,N,N-trimethyl-5-aminovaleric acid, a reagent capable of reducing or preventing the production of N,N,N-trimethyl-5-aminovaleric acid, and a reagent capable of promoting the metabolism of N,N,N-trimethyl-5-aminovaleric acid in vivo.

3. The use according to claim 1, wherein the fatty liver comprises non-alcoholic fatty liver disease; and the heart failure is heart failure caused by myocardial hypertrophy.

4. The use according to claim 3, wherein the non-alcoholic fatty liver disease comprises high-fat diet-induced non-alcoholic fatty liver disease; and the myocardial hypertrophy comprises high-fat diet-induced myocardial hypertrophy.

5. A system for diagnosing fatty liver or heart failure, wherein the system comprises a test device, a computing device and an output device; the test device comprises a sampler and a tester, the sampler is used to collect a sample to be tested, and the tester is used to test the concentration of N,N,N-trimethyl-5-aminovaleric acid in the sample to be tested; the computing device comprises a memory and a processor, the memory stores a computer program, and the processor is configured to execute the computer program stored in the memory to implement the following determination:
if the test result of the concentration of N,N,N-trimethyl-5-aminovaleric acid in the sample to be tested is 0.576-100 μmol/L, it is determined that the subject corresponding to the sample to be tested is a fatty liver patient or a heart failure patient.

6. The system according to claim 5, wherein the fatty liver comprises non-alcoholic fatty liver disease; and the heart failure is heart failure caused by myocardial hypertrophy.

7. The system according to claim 6, wherein the non-alcoholic fatty liver disease comprises high-fat diet-induced non-alcoholic fatty liver disease; and the myocardial hypertrophy comprises high-fat diet-induced myocardial hypertrophy.

8. The system according to claim 5, wherein the sample to be tested comprises a plasma sample and/or a serum sample.

9. The system according to claim 5, wherein the test device comprises a mass spectrometer or a liquid chromatograph-mass spectrometer.

10. The system according to any one of claims 5-9, wherein the output device is used to output the test result of the test device or the determination result of the computing device, and the output device comprises at least one of a display, a printer and an audio output device; and the computing device comprises at least one of a computer host, a central processing unit and a network server.
